(19) United States
(12) Patent Application Publication
(10) Pub. No.: US 2014/0142376 A1
(43) Pub. Date: May 22, 2014

(54) MEDICAL SYSTEMS AND METHODS

(71) Applicant: Somnath GHOSH, Medford, OR (US)

(72) Inventor: Somnath GHOSH, Medford, OR (US)

(21) Appl. No.: 14/084,486

(22) Filed: Nov. 19, 2013

Related U.S. Application Data
(60) Provisional application No. 61/728,170, filed on Nov. 19, 2012.

(51) Int. Cl. A61M 1/00; A61B 1/04; A61M 25/10; A61B 1/018; A61B 1/267

(52) U.S. Cl. CPC A61M 1/0062; A61B 1/018; A61B 1/2676; A61M 25/10184; A61B 1/04; A61M 25/10182
USPC 600/104

(57) ABSTRACT

A medical device for performing a bronchoalveolar lavage (BAL) that includes a tubing having a first lumen configured for communication with a first reservoir, the first lumen having an open distal end configured for communication with the first reservoir, and a second lumen that is separate from the first lumen and configured for communication with a second reservoir. The tubing also includes an inflatable cuff that is disposed proximate the open distal end and configured for communication with the second reservoir via the second lumen, wherein the open distal end and the inflatable cuff in a deflated position are both configured for receipt within a working channel of a bronchoscope.

### CROSS-REFERENCE TO RELATED APPLICATIONS

This application claims priority to U.S. Provisional Patent Application Ser. No. 61/728,170, filed Nov. 19, 2012, and entitled Medical Systems and Methods, the disclosure of which is incorporated herein by reference.

### INTRODUCTION

The present disclosure is directed to medical systems and methods, and particularly systems and methods related to the bronchoalveolar lavage (BAL) procedure and/or an endobronchial balloon tamponade of pulmonary hemorrhage.

The BAL procedure, performed during flexible bronchoscopy, is a method to learn information about immunologic, inflammatory, and infectious processes occurring in the lungs, particularly at the alveolar level. The BAL procedure has been a useful diagnostic and therapeutic tool ever since flexible bronchoscopy revolutionized the field of pulmonary medicine. The BAL procedure can be used on any subject, including humans and animals.

The current BAL procedure typically involves navigating a bronchoscope to a desired segment/subsegment (limited by “reach”) of the lung, “wedging” the bronchoscope (which entails jamming the scope against the lung walls, hopefully to create a waterproof seal), and then infusing/instilling and then retracting/aspirating lavage fluid for diagnostic analysis.

However, there is no general consensus about what the optimal BAL technique is. For example, there is a lack of consensus regarding the optimal volume of infused saline, amount of suction applied, etc. Moreover, the current ways of performing the BAL procedure may have one or more of the following drawbacks/difficulties: (A) inadequate “wedging,” which can result in leakage of lavage fluid and also “overwedging,” which can result in distal airway collapse and resultant diminished fluid recovery; (B) airway trauma as a result of wedging and overwedging; (C) inability to wedge in the desired subsegment due to size concerns; (D) poor return of lavage fluid for diagnostic analysis; (E) loss of wedge as a result of coughing or bronchoscopic movements; and/or (F) contamination with upper airway organisms.

The following patents include information about prior art BAL systems and methods: U.S. Pat. Nos. 5,135,490 and 5,246,012, both of which are hereby incorporated by reference.

### BRIEF DESCRIPTION OF THE DRAWINGS

FIG. 1 shows a schematic illustration of an embodiment of a medical device according to the present disclosure.

FIG. 2 shows an embodiment of the medical device including a tube portion with reference markings and an inflatable cuff.

FIG. 3 shows a cross-section of the tube portion taken along the line 3-3 in FIG. 2.

FIG. 4 shows the embodiment of FIG. 2 with a portion of the tube portion disposed inside a working channel of a bronchoscope, and the bronchoscope inserted into an air passageway of a subject.

FIG. 5 is a detailed view taken at 5 in FIG. 4, showing the bronchoscope positioned in the air passageway, the inflatable cuff and a distal end of the tubing portion disposed inside the working channel of the bronchoscope, and the inflatable cuff in a deflated position.

FIG. 6 is a detailed view similar to FIG. 5, but showing the tubing portion extended to a desired position beyond a distal tip of the bronchoscope.

FIG. 7 is a detailed view similar to FIG. 6, but showing the inflatable cuff in an inflated position to create a substantially sealed-off area in the air passageway.

FIG. 8 shows an embodiment of a method of the present disclosure for performing a BAL procedure.

FIG. 9 shows an embodiment of a method of the present disclosure for an endobronchial balloon tamponade of pulmonary hemorrhage.

### DETAILED DESCRIPTION

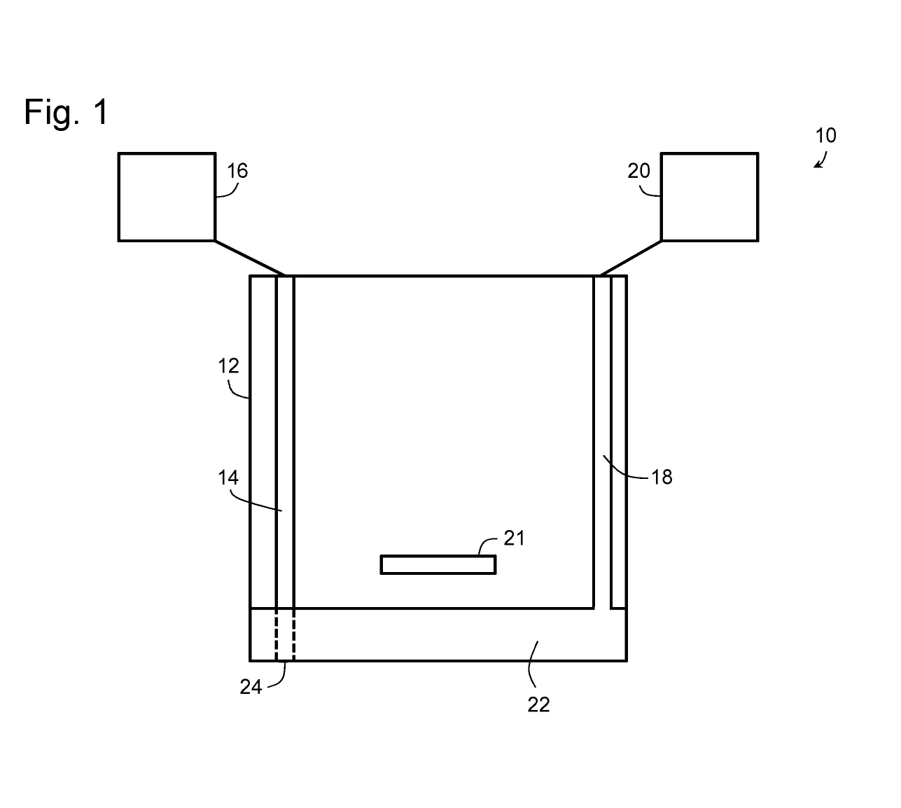

Turning to FIG. 1, a medical device 10 of the present disclosure may include a tubing 12 having a first lumen (or channel) 14 in communication with a fluid source (or reservoir) 16, a second lumen (or channel) 18 in communication with an air (or gas or fluid) source (or reservoir) 20, one or more reference markings 21 disposed on tubing 12, an inflatable portion 22 in communication with air source 20 via second lumen 18, and an open distal end 24 in communication with fluid source 16 via first lumen 14. Inflatable portion 22 may be disposed on tubing 12 proximate open distal end 24, but may be disposed in any suitable position on tubing 12. Air (or gas or fluid) source 20 can be used to inflate inflatable portion 22, and fluid source 16 may be used to instill and/or aspirate fluid through open distal end 24. All of, or a portion of, tubing 12 and/or reference markings 21 and/or inflatable portion 22 may be configured to fit within a working channel of a bronchoscope.

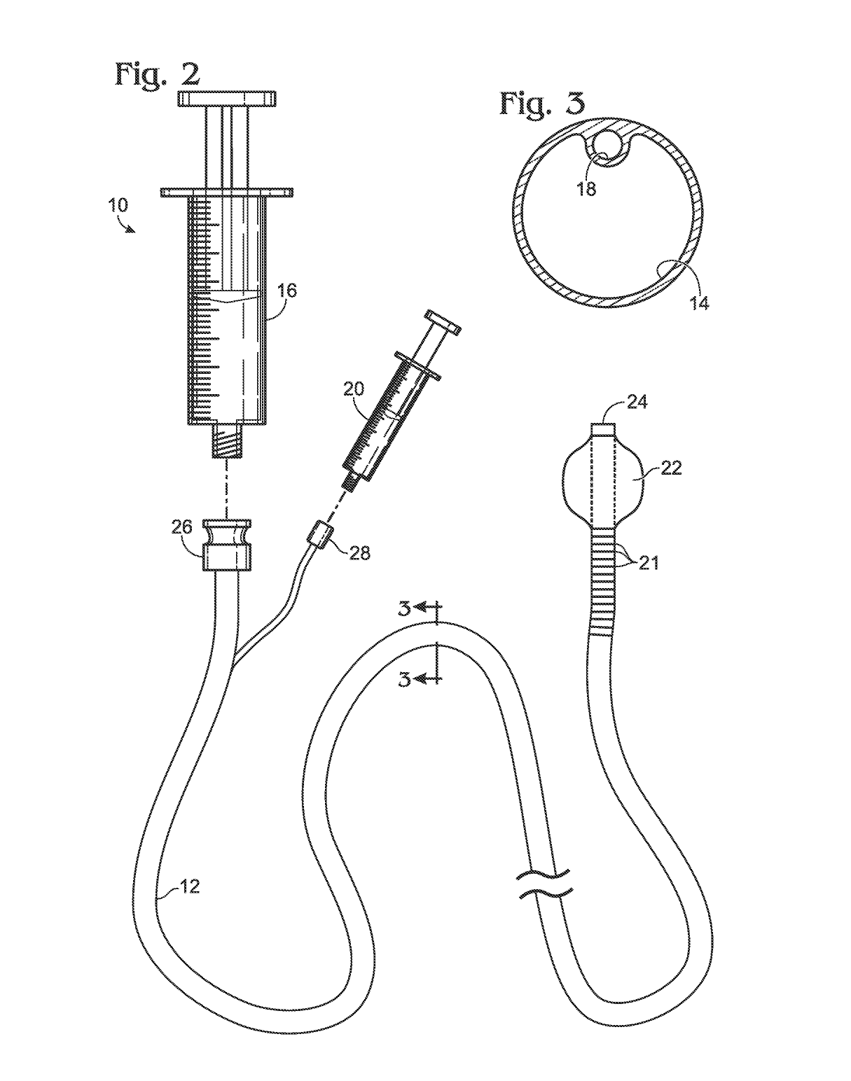

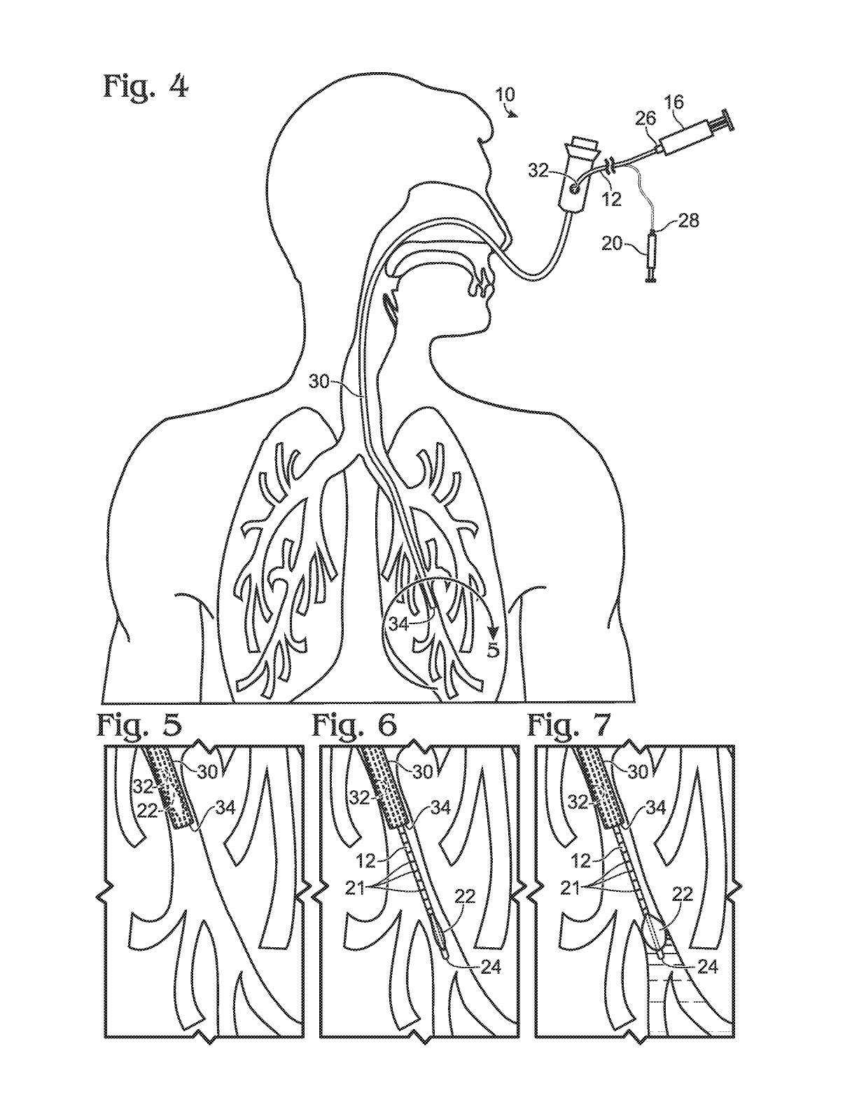

FIG. 2. shows an embodiment of medical device 10, in which medical device 10 includes a catheter design that includes tubing 12 with a first connector 26 for attaching a fluid source 16 (e.g., a fluid syringe housing fluid), a first lumen 14 (shown in FIG. 3) for carrying fluid in communication with fluid source 16, a second connector 28 for attaching an air source 20 (e.g., an air syringe housing air), a second lumen 18 (also shown in FIG. 3) for carrying air that is in communication with air source 20 (e.g. air syringe), one or more reference markings 21, and an inflatable cuff 22 (shown in an inflated state or position in FIGS. 2 and 7, and shown in a deflated state or position in FIGS. 5 and 6). As shown in FIG. 3, second lumen 18 may be separate from first lumen 14.

Tubing 12 may be made of a clear plastic or other synthetic materials. Tubing 12 may have a rigidity comparable to an outer covering of currently used bronchial cytology brushes. Any type of tubing suitable for a BAL procedure and/or a balloon tamponade for pulmonary hemorrhage can be used, as the case may be.

Both first and second connectors 26, 28 may be located at or near a proximal end of tubing 12, but may be located at any suitable position(s). First connector 26 may connect the syringe containing a fluid (e.g., normal saline) to be infused. Second connector 28 may connect the syringe housing air with inflatable cuff 22. The first and second connectors can be any type of connector suitable for respectively connecting fluid and air/gas sources. For example, the connectors may allow for connecting syringes filled with fluid and/or air. A stop-cock system may also be employed to control the flow of air and/or fluid between the tubing and the respective air and fluid sources. As shown, the first connector and/or the second connector may be twist type connectors, onto which the syringes may twist. The first connector may be larger than the second connector, though this is not required.

One or more reference markings 21 may be disposed on tubing 12 leading up to inflatable cuff 22 (see FIGS. 2 and 5). The markings may be visible using a camera associated with the bronchoscope while positioning tubing 12 in a lung area being examined. One or more reference markings 21 may be disposed on either or both sides of cuff 22, in any configuration and/or spaced apart in any way desired.

In some embodiments, the markings may be ½ centimeter apart, though the markings may be spaced apart in any distances desired. In some embodiments, there may be 10 spaced-apart markings, though any number of markings desired may be used. In some embodiments, the most distal marking may indicate the lowest number while the most proximal marking may indicate the highest number. For example, from most distal marking to most proximal marking, the markings may be indicated as follows: 0.5 cm/1.0 cm/1.5 cm/2.0 cm/2.5 cm/3.0 cm/3.5 cm/4.0 cm/4.5 cm/5.0 cm (or 1/2/3/4/5/6/7/8/9/10, etc.). These markings may correspond to indicate the distance beyond the end of the bronchoscope that the distal end of the tubing is extending. These markings may correspond to indicate the distance beyond the end of the bronchoscope that the inflatable cuff is extending. These markings may correspond to indicate any other distance desired to be indicated during a BAL procedure and/or for a balloon tamponade for pulmonary hemorrhage, as the case may be.

In some embodiments of the present disclosure, an indication and/or an identification of a distance that tubing 12 extends beyond distal tip 34 of bronchoscope 30 (see FIGS. 6 and 7) may be provided by any suitable structure, mechanism, and/or apparatus other than, or in addition to, markings 21. For example, device 10 may include an apparatus for digitally calculating the distance that tubing 12 extends beyond distal tip 34. For example, device 10 (or a system associated with device 10) may include a computer running a software application configured to detect the distance that tubing 12 extends beyond distal tip 34 (or any other suitable extension reference point). For example, inflatable cuff 22 may have a known diameter (e.g., deflated state diameter). As inflatable cuff 22 moves away from distal tip 34, the known diameter may appear smaller in an image acquired by the camera associated with bronchoscope 30, and the software application may be configured to calculate the distance by comparing the apparent smaller diameter to the known diameter.

Inflatable portion or cuff 22 may be configured to wrap around a portion of, or the entire circumference of, tubing 12. Air (or other suitable matter) from reservoir 20 may be used to insufflate (or inflate) inflatable cuff 22 via lumen 18. Inflatable cuff 22 when insufflated may expand around all or a portion of the circumference of tubing 12, thereby securing tubing 12 in a desired position against the lung walls. The desired position may create a sealed chamber of the area being examined that is in communication with open distal end 24 of tubing 12. The inflatable cuff may be configured so that in a deflated state it would blend with the surface of the rest of the catheter tubing, thereby not using up precious space in the working channel of the bronchoscope. In some embodiments, inflatable cuff 22 in the deflated state may have an outer circumference that is substantially equal to (or just slightly larger than) an outer circumference of first lumen 14 in a region of tubing 12 proximal inflatable cuff 22. The inflatable cuff may be made of rubber, silicone or other appropriate material. The inflatable cuff may be configured similar to a balloon of a pulmonary artery catheter. The inflatable cuff may take any form desired, so long as it is capable of creating a sealed chamber in communication with the open distal end of the tubing.

In some embodiments, source 20 may be a source for matter other than air, which may be suitable for expanding cuff 22. For example, source 20 may be a source of a suitable gas or fluid, such as water, which may be used to selectively fill cuff 22.

Now turning to FIGS. 4-7, tubing 12 may be configured to interact with a bronchoscope 30, such as a flexible bronchoscope. For example, tubing 12 may be configured for insertion into a working channel 32 of bronchoscope 30. Tubing 12 and/or markings 21 and/or inflatable portion 22 may be configured to fit within working channel 32 without extending beyond the outermost open edge of distal tip 34 until bronchoscope 30 is positioned at a suitable location within the air passageway of the subject, as shown in FIGS. 4 and 5. Distal tip 34 of the bronchoscope may include an image acquisition portion of the camera associated with bronchoscope 30, which may allow identification of the suitable location in which to position distal tip 34 of bronchoscope 30. As shown in FIG. 5, inflatable portion 22 may fit inside working channel 32 when inflatable portion 22 is in the deflated position.

Once bronchoscope 30 has been positioned in the air passageway of the subject (e.g., in a bronchial segment), tubing 12 may be extended beyond positioned distal tip 34 to a desired position (e.g., in a sub-segment of the bronchial segment), as shown in FIG. 6. For example, markers 21 may be viewed through the camera associated with bronchoscope 30 as open distal end 24 is extended past distal tip 34, which may provide an indication of an extended distance of open distal end 24 past distal tip 34. In some embodiments, the desired position of tubing 12 (e.g., open distal end 24 and/or inflatable portion 22) may correspond to a particular extension of tubing 12 past distal tip 34. In some embodiments, the desired position may correspond to a particular location in the air passageway (e.g., a location viewed via the camera associated with bronchoscope 30).

As shown in FIG. 7, once tubing 12 has been positioned in the desired location, cuff 22 may be insufflated to create a substantially sealed-off area in the air passageway of the subject (e.g., in the bronchial sub-segment). For example, cuff 22 may be insufflated with air (or other suitable matter) from source 20 (see FIG. 4) via lumen 18 (see FIGS. 1 and 3).

Once cuff 22 has been insufflated to create the substantially sealed-off area, fluid from fluid source 16 (see FIG. 4) may be injected into the substantially sealed-off area, as shown in FIG. 7. For example, fluid from source 16 may be injected into the substantially sealed-off area through open distal end 24 via lumen 14 (see FIGS. 1 and 3).

Fluid may be aspirated (or removed) from the substantially sealed-off area (e.g., via open distal end 24, and lumen 14) to produce a fluid specimen from the substantially sealed-off area. In some embodiments, the fluid specimen may be aspirated into reservoir 16.

In some embodiments, the fluid specimen (or other specimen and/or fluid) may be aspirated from the substantially sealed-off area via open distal end 24 and lumen 14 without fluid first being injected (or instilled) into the substantially sealed-off area. For example, the fluid specimen (or other specimen and/or fluid) may not include components introduced by device 10 (e.g., fluid from reservoir 16), and/or the fluid specimen (or substance, such as blood) may have been present in the air passageway prior to tubing 12 being positioned in the air passageway.

There are a wide variety of bronchoscopes with available lumen/working channel sizes that can be used with tubing 12. For example, OLYMPUS® makes fiber optic scopes with working channel sizes from 1.2 mm to 3 mm. The most widely used scopes have working channel sizes of 2.0 mm or 2.2 mm. Ensuring that tube 12 is sized to fit into this range of working channels may ensure there is an adequate suction channel.

One or more of the following components may also be included, or used in connection with, medical device 10: a sterile collection trap, sterile saline, a vacuum source, a syringe, a stop-cock, such as a 3-way stop-cock, and/or lidocaine 1-2%. Intubation and ventilation equipment may also be employed as oxygenation may deteriorate during the BAL procedure and/or the balloon tamponade for pulmonary hemorrhage, as the case may be. This risk of hypoxia may not be more than that encountered in the traditional techniques and oxygenation and ventilation is dependent on the airway around the bronchoscope and not the working channel.

BAL

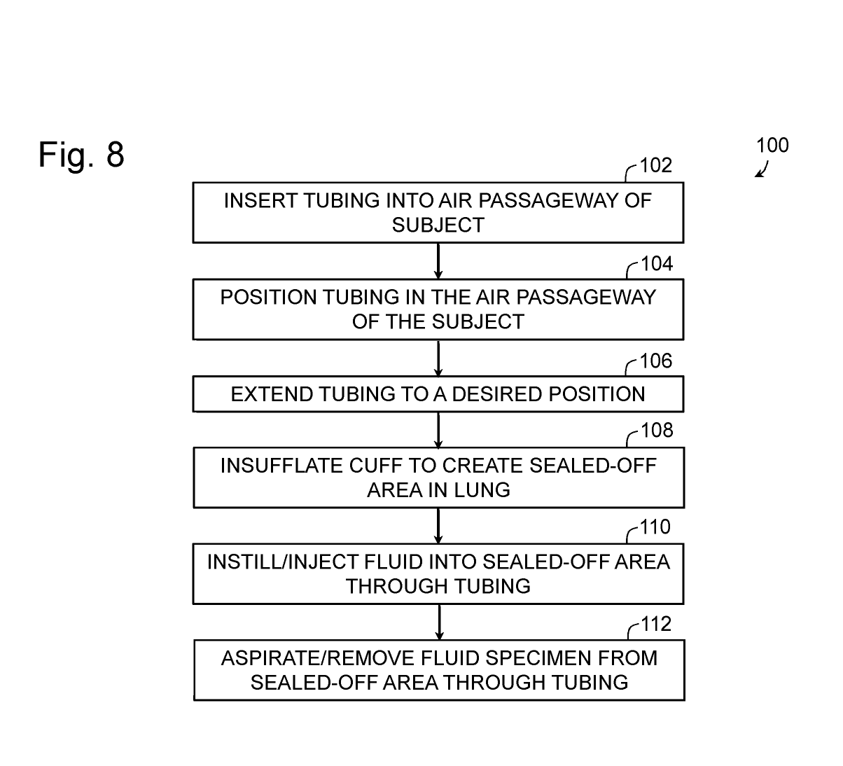

FIG. 8 shows an embodiment of a method 100 of using a medical device in a BAL procedure. A medical device may be provided as described above. For example, the tubing may be connected to an air source and a fluid source. A portion of the tubing may then be extended into a working channel of a bronchoscope.

Method 100 may include a step 102 of inserting the tubing into an air passageway of a subject. The tubing may be a movable tubing with an inflatable cuff in a deflated position. In some embodiments, step 102 may involve inserting the bronchoscope housing the tubing into the air passageway of the subject. The tubing may be housed in the working channel of the bronchoscope.

In some embodiments, it may be desired that the distal end of tubing remains inside the working channel such that no portion of tubing (including the inflatable cuff and markings) extends outside the outermost edge of the distal tip of the bronchoscope into the lungs while the bronchoscope is being positioned. This could help prevent contamination of the tubing by fluid in the upper passageways of the lungs of the subject. In some embodiments, the catheter (e.g., tubing) may be inserted only after the bronchoscope is advanced till the point that the desired subsegmental bronchus is visible.

Method 100 may include a step 104 of positioning the tubing in the air passageway of the subject (e.g., as desired). For example, step 104 may involve positioning the movable tubing in the air passageway of the subject. For example, step 104 may involve positioning the bronchoscope housing the tubing in the air passageway of the subject. For example, the bronchoscope can be guided and positioned in the desired subsegment. The distal tip of the bronchoscope may be positioned at the desired subsegment without traumatizing the airway.

Method 100 may include a step 106 of extending the tubing to a desired position. For example, step 106 may involve extending the tubing beyond the positioned distal tip of the bronchoscope to a desired position. For example, once the bronchoscope is positioned, then the tubing can be extended beyond the distal end of the bronchoscope and advanced to the desired airway that is the target of the analysis. The reference markings (e.g., depth markers) on the tubing may provide an idea about how far the distal end is advanced (e.g., extended beyond the distal end of the bronchoscope). For example, the distal end and the inflatable cuff of the tubing may first advance out from the tip of the bronchoscope, followed by the depth markings on the tubing. As the tube is advanced forward toward the desired airway that is the target of the analysis, the depth markings may provide an indication (e.g., using the camera images of the camera associated with the bronchoscope) regarding how far the distal end of the tubing has advanced. This may help the practitioner position the distal end of the catheter into a position for proper wedging and to avoid inadequate wedging and overwedging.

Method 100 may include a step 108 of insufflating (or inflating, or filling) the cuff to create a substantially sealed-off area in a lung of the subject. For example, once the tubing is positioned as desired, then the cuff may be insufflated to create an adequate wedge having a snug air and water seal (e.g., between the cuff and a wall of the lung segment or subsegment). The cuff may be insufflated with air from air source 20. For example, a syringe attached to the second connector may be used to insufflate the wedge. The practitioner can watch the insufflation occur using the camera associated with the bronchoscope to ensure there is an adequate and snug seal created. The insufflated wedge may create a sealed area of the lung that is in communication with the open distal end of the tubing.

Method 100 may include a step 110 of instilling/injecting fluid into the sealed-off area through the tubing. For example, once the cuff is insufflated, then fluid may be instilled (or injected or infused) through the tubing into the sealed area of the lung being analyzed. The fluid may be a saline solution. The fluid may be instilled using fluid source 16. For example, the saline may be instilled by depressing the fluid syringe into the tubing.

Method 100 may include a step 112 of aspirating/removing a fluid specimen from the sealed-off area through the tubing. For example, the fluid may be aspirated (or removed) from the sealed area being analyzed to produce a lavage fluid specimen.

In some embodiments, the lavage fluid specimen may be aspirated using the same fluid source 16, such as the same syringe, that was used to instill the saline to obtain an optimally pure sample. While the wedge is maintained, the lavage fluid specimen may be recovered immediately into the same fluid source (e.g. syringe) using a suction of approximately 50-80 mmHg. The suction at least may be gentle enough that visible airway collapse does not occur.

When no further fluid can be aspirated, then a second saline filled syringe (or the same syringe refilled) may then be attached to the tubing and the procedure repeated. A clamp or stopcock may be closed, allowing removal of a syringe. These steps may be repeated as necessary. For example, it may be necessary to instill a total of approximately 100-240 mL of saline in multiple aliquots (e.g., of approximately 20-60 ml each) in order to obtain an adequate lavage fluid specimen (approximately 40-60 mL—usually 40-70% recovery of total instillate). Higher (or lower) aliquots and higher (or lower) total volume can be used as desired. Since embodiments of the present disclosure (e.g., medical device 10) may allow for farther ‘reach’ (e.g., past the distal tip of the bronchoscope and toward a distal portion of the lung) smaller aliquots and lower total volume may be needed which may be one advantage of an embodiment of a medical device according to the present disclosure.

After all desired lavage fluid samples are retrieved, then inflatable cuff 22 may be deflated. The deflated cuff and open distal end 24 of the tubing may then be completely retrieved into, and out of working channel 32 of bronchoscope 30. The bronchoscope may then be removed from the subject when deemed appropriate.

Performing the BAL procedure using the systems and methods of embodiments of the present disclosure may include one or more of the following advantages over existing BAL procedures and systems: (A) may minimize airway trauma; (B) may have the ability to direct the BAL catheter tubing to a more selective and peripheral airway; (C) may buffer the effects of coughing by maintaining the wedge; (D) may enhance return; (E) may provide a more “pure alveolar sample”; (F) because the catheter ventures beyond a bronchoscope, the amount of lung sampled may be smaller (and more peripheral), hence a smaller volume of normal saline may be required to be infused in order to obtain a good sample, which may minimize the iatrogenic dead space and potential post-procedure hypoxia, and may also minimize any volume burden in patients with fragile hemodynamics; (G) a relatively inexpensive disposable catheter may provide the above advantages for a widely used procedure that has major potential therapeutic consequences; and/or (H) considering the diagnostic value of these procedures, especially in the light of their tremendous volume, the use of a catheter according to aspects of the present disclosure may significantly enhance diagnostic yield and patient safety for BAL procedures.

### Balloon Tamponade for Pulmonary Hemorrhage

Another possible use of medical device 10 of the present disclosure may be for use in systems and methods related to a balloon tamponade for pulmonary hemorrhage. A medical device may be provided as described above. In some embodiments, the medical device may only need to be connected to air source 20 for this procedure. In some embodiments, the medical device may not require the use of reference markings 21 for this procedure. In some embodiments, the length of the tubing may be longer then the length of the tubing for the BAL procedure.

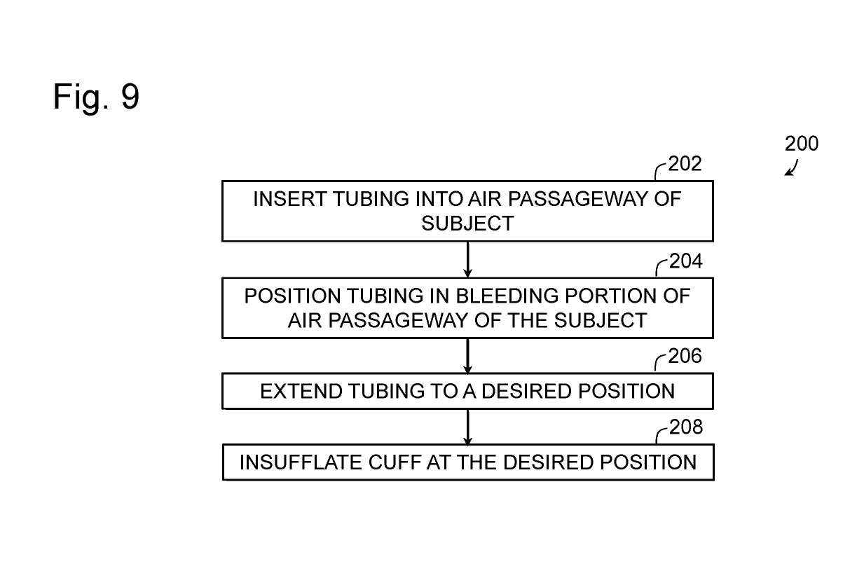

FIG. 9 illustrates a method 200 for performing an endobronchial balloon tamponade of pulmonary hemorrhage according to aspects of the present disclosure. One or more aspects of the method described below may be similar to the BAL method described above, except where differences are indicated.

Method 200 may include a step 202 of inserting tubing into an air passageway of a subject. Step 202 may involve inserting a bronchoscope housing the tubing into the air passageway of the subject. For example, the bronchoscope may have a working channel, and the tubing may be housed and movable in the working channel.

Method 200 may include a step 204 of positioning the tubing in a bleeding portion of the air passageway of the subject. For example, step 204 may involve positioning the bronchoscope (housing the tubing) in the bleeding portion of the air passageway of the subject.

Method 200 may include a step 206 of extending the tubing to a desired position. For example, step 206 may involve extending the tubing beyond the positioned distal tip of the bronchoscope to a desired position in the air passageway of the subject.

Method 200 may include a step 208 of insufflating a cuff at the desired position (e.g., insufflating an inflatable cuff positioned proximate a distal end of the tubing). For example, step 208 may involve insufflating in the air passageway the inflatable cuff positioned proximate the distal end of the tubing.

Traditionally, if significant bleeding occurs during an interventional procedure (e.g., endobronchial biopsy, brush cytology, needle biopsy, etc.) or spontaneously due to any pathological process, some measures currently being used include instillation of ice-cold lavage, epinephrine, etc. If these methods prove insufficient, the bronchoscope is advanced and wedged into the bleeding airway. The wedge is maintained until hemostasis is achieved. The bronchoscope at this point is being used purely as an occluder. The camera is rendered useless because of the blood. It then becomes a matter of ‘waiting and watching’ and hoping for hemostasis. Backup from other specialties (thoracic surgery/anesthesiology/interventional radiology) is usually solicited. If bleeding is torrential, single lung intubation is performed. This involves advancing an endotracheal tube into the mainstem bronchus of the healthy lung and inflating the cuff so that the bleeding lung is isolated from the healthy lung and oxygenation and ventilation can continue. In life threatening pulmonary hemorrhages, it is seldom the blood loss itself that proves fatal. It is usually the dead space created by the blood and the resultant hypoxia that kills the subject.

The medical device or catheter 10 of the present disclosure may be useful in balloon tamponade for pulmonary hemorrhage, such as in the above situations. For example, once significant bleeding is encountered, and if for example, ice-cold saline or epinephrine cannot achieve hemostasis, the catheter 10 may be introduced into the bleeding segment or subsegment and the cuff 22 inflated. In some embodiments, catheter 10 may be introduced at any point desired into the bleeding segment or subsegment and the cuff 22 inflated. Depending on the nature of the hemorrhage, catheter 10 may be deflated shortly thereafter to check for hemostasis, or left longer. If it is suspected that it will need to be in there (e.g., at the desired point) longer, the end of the catheter attached to the syringes outside the bronchoscope may be clamped with a clamping device (e.g, forceps, etc.). The bronchoscope may then be removed over the catheter. Hence the catheter used for this purpose may be longer than the one used for the BAL to allow for the length of the bronchoscope. Once the bronchoscope is out of the patient's airway, another clamping device (e.g., forceps, etc.) may then be used to clamp the catheter beyond the bronchoscope tip. The clamping may be to make sure the cuff remains inflated. The patient's airway is thus free of the bronchoscope and ventilation and oxygenation may continue with only the catheter and inflatable cuff (e.g., in the inflated state) in place. Once it is felt that the hemorrhage has stopped, or after other interventions have determined and addressed the source of the bleed (e.g., interventional radiology assisted embolization of the particular segmental vasculature) the cuff may be deflated and the catheter removed. In some embodiments, the cathether may be deflated under bronchoscopic view by unclamping the clamping devices (e.g. forceps) and the catheter removed. If bleeding recurs, another catheter may be immediately deployed.

Using the systems and methods of embodiments of the present disclosure for a balloon tamponade for pulmonary hemorrhage may include one or more of the following advantages over existing procedures and systems: (A) may have less dead space (as only a segment or subsegment will be occluded), which may prove life-saving in patients in whom single lung ventilation may not be sufficient; (B) may have less blood loss as the culprit airway is tamponaded closer to the bleeding source; (C) blood is an airway irritant, and when selective intubation of the healthy airway is performed, the blood may be free to seep into all the other airways of the affected lung, thus the patient's entire lung may fill with blood and the accompanying inflammation may be substantial (if not fatal), which may be minimized with the use of the catheter; (D) the catheter may be left in the patient as long as required while still allowing spontaneous or assisted ventilation; and/or (E) direct visualization of the airway while deflating the cuff may identify continued bleeding and may be immediately addressed by deploying another catheter.

The following paragraphs may describe one or more embodiments according to the present disclosure.
A. A medical device for performing a bronchoalveolar lavage (BAL), comprising: a tubing having a first lumen configured to connect to a fluid source, the first lumen having an open distal end, a second lumen configured to connect to an air source, an inflatable cuff that is disposed proximate the open distal end and connected to the second lumen, and one or more distance markers disposed on the tubing.
B. The medical device of paragraph A, further comprising a bronchoscope that has a working channel sized to house a portion of the tubing.
C. The medical device of paragraph B, wherein the working channel is sized to house a portion of the tubing that includes the inflatable cuff while the cuff is in a deflated state.
D. The medical device of paragraph A, further comprising a bronchoscope that has a working channel sized to house a portion of the tubing that includes the inflatable cuff.
E. The medical device of paragraph A, wherein a plurality of markers are disposed on the tubing, spaced apart from each other by 0.5 cm.
F. The medical device of paragraph A, wherein the markers are disposed on the tubing on a proximal side of the inflatable cuff in a direction toward a proximal end of the tubing.
G. The medical device of paragraph A, wherein the fluid source is a syringe housing fluid and the air source is a syringe housing air.
H. A method for performing a bronchoalveolar lavage (BAL), comprising:

inserting a bronchoscope with a working channel housing a movable tubing into an air passageway of a subject;

positioning the bronchoscope in the air passageway;

extending the tubing beyond a positioned distal tip of the bronchoscope to a desired position by using depth marker indications on the tubing;

insufflating an inflatable cuff positioned proximate a distal end of the tubing to create a wedge to seal off an area of the lung for analysis

instilling fluid into the sealed-off area through the tubing; and

aspirating a lavage fluid specimen from the sealed-off area through the tubing.
I. The method of paragraph H, further comprising instilling approximately 20-40 ml of fluid.
J. The method of paragraph I, further comprising repeating the instilling and aspirating steps at least 4 times.
K. The method of paragraph H, further comprising deflating the cuff and completely retracting the distal end of the tubing and the cuff into the working channel from the air passageway.
L. The method of paragraph H, further comprising extending the cuff from a deflated state inside the working channel to outside the working channel toward the desired position and insufflating the cuff at the desired position to an inflated state.
M. The method of paragraph L, further comprising extending a first marker of a plurality of markers from inside the working channel to a distal position outside the working channel after the cuff is extended outside the working channel.
N. The method of paragraph H, further comprising extending a first marker from inside the working channel to a distal position outside the working channel.
O. The method of paragraph N, further comprising extending a second marker from inside the working channel to a distal position outside the working channel after the first marker was extended.
P. A medical device for performing an endobronchial balloon tamponade of pulmonary hemorrhage, comprising: a tubing having a lumen configured to connect to an air source and an inflatable cuff that is disposed proximate the open distal end and connected to the lumen.
Q. The medical device of paragraph P, further comprising a bronchoscope that has a working channel sized to house a portion of the tubing.
R. The medical device of paragraph Q, wherein the length of the tubing is longer than the length of the working channel.
S. The medical device of paragraph Q, wherein the working channel is sized to house a portion of the tubing that includes the inflatable cuff while the cuff is in a deflated state.
T. The medical device of paragraph P, further comprising a bronchoscope that has a working channel sized to house a portion of the tubing that includes the inflatable cuff.
U. The medical device of paragraph P, wherein the air source is a syringe housing air.
V. A method for performing an endobronchial balloon tamponade of pulmonary hemorrhage, comprising:

inserting a bronchoscope with a working channel housing a movable tubing into an air passageway of a subject;

positioning the bronchoscope in the air passageway;

extending the tubing beyond a positioned distal tip of the bronchoscope to a desired position; and

insufflating an inflatable cuff positioned proximate a distal end of the tubing.
W. The method of paragraph V, further comprising positioning the bronchoscope in a bleeding segment or subsegment of the air passageway and extending the tubing to the desired position in the bleeding segment or subsegment.
X. The method of paragraph V, further comprising deflating the inflatable cuff to check for hemostasis using a camera of the bronchoscope.
Y. The method of paragraph V, further comprising clamping an end of the medical device that is outside the bronchoscope and removing the bronchoscope from the air passageway while leaving the tubing in the air passageway.
Z. The method of paragraph Y, further comprising clamping the tubing at a point beyond the bronchoscope tip after the bronchoscope is removed from the air passageway to ensure the inflatable cuff remains inflated.
AA. The method of paragraph Y, further comprising unclamping the end to deflate the inflatable cuff.
BB. The method of paragraph Z, further comprising unclamping the end and the point beyond the bronchoscope tip to deflate the inflatable cuff.
CC. The method of paragraph Z, further comprising deflating the cuff and visually confirming the cuff is sufficiently deflated for removal from the air passageway by using the camera of the bronchoscope.
DD. The method of paragraph V, further comprising inserting a second tubing if bleeding recurs after removal of the tubing from the air passageway.
EE. A medical device for performing a bronchoalveolar lavage (BAL), comprising:

a tubing having
a first lumen in communication with a first reservoir, the first lumen having an open distal end in communication with the first reservoir,
a second lumen that is separate from the first lumen and in communication with a second reservoir, and
an inflatable cuff that is disposed proximate the open distal end and in communication with the second reservoir via the second lumen, wherein the open distal end and the inflatable cuff in a deflated position are both configured for receipt within a working channel of a bronchoscope
FF. The medical device of paragraph EE, wherein the first reservoir is a fluid source housing fluid and the second reservoir is an air source housing air.
GG. The medical device of paragraph EE, wherein a portion of the tubing including the open distal end and the inflatable cuff is sized to be housed within the working channel of the bronchoscope while the cuff in the deflated position.
HH. The medical device of paragraph EE, further comprising one or more distance markers disposed on the tubing.
II. The medical device of paragraph HH, wherein the markers are disposed on the tubing on a proximal side of the inflatable cuff in a direction toward a proximal end of the tubing.
JJ. The medical device of paragraph II, wherein the plurality of markers are spaced apart from each other on the tubing in intervals of about 0.5 centimeters (cm).
KK. A method for performing a bronchoalveolar lavage (BAL), comprising:

inserting a movable tubing with an inflatable cuff in a deflated position into an air passageway of a subject;

positioning the movable tubing in the air passageway by visually monitoring an image of a distal portion of the tubing inserted into the air passageway;

insufflating the inflatable cuff to an inflated position to create a wedge to seal off an area of the air passageway; and

aspirating a lavage fluid specimen from the sealed-off area through the tubing.
LL. The method of paragraph KK, further comprising instilling fluid into the sealed-off area through the tubing before aspirating the lavage fluid specimen.
MM. The method of paragraph LL, further comprising instilling approximately 20 milliliters (ml) to approximately 40 ml of fluid and repeating the instilling and aspirating steps at least 4 times.
NN. The method of paragraph LL, further comprising inserting the movable tubing into the air passageway of the subject within a working channel of a bronchoscope and positioning the bronchoscope in the air passageway.
OO. The method of paragraph NN, further comprising extending the inflatable cuff in the deflated position to a desired position beyond a positioned distal tip of the bronchoscope and insufflating the inflatable cuff, which is disposed proximate a distal end of the tubing, to the inflated position to create the wedge.
PP. The method of paragraph OO, further comprising using a camera associated with the bronchoscope to confirm proper positioning of the cuff in the air passageway, the camera transmitting a still image or video image for visual monitoring.
QQ. The method of paragraph NN, further comprising deflating the cuff and completely retracting the distal end of the tubing and the cuff into the working channel from the air passageway before removing the bronchoscope after aspiration.
RR. The method of paragraph NN, further comprising extending a first depth marker of a plurality of depth markers on the tubing from inside the working channel to a distal position outside the working channel after the cuff is extended outside the working channel.
SS. The method of paragraph RR, further comprising using a camera to observe the first depth maker to confirm proper positioning of the cuff in the air passageway.
TT. A method for performing an endobronchial balloon tamponade of pulmonary hemorrhage, comprising:

inserting a bronchoscope with a working channel housing a movable tubing into an air passageway of a subject;

positioning the bronchoscope in the air passageway;

extending the tubing beyond a positioned distal tip of the bronchoscope to a desired position in the air passageway; and

insufflating in the air passageway an inflatable cuff positioned proximate a distal end of the tubing.
UU. The method of paragraph TT, further comprising positioning the bronchoscope in a bleeding segment or subsegment of the air passageway and extending the tubing to the desired position in the bleeding segment or subsegment.
VV. The method of paragraph TT, further comprising deflating the inflatable cuff to check for hemostasis using a camera of the bronchoscope.
WW. The method of paragraph TT, further comprising removing the bronchoscope from the air passageway while leaving the tubing and the insufflated cuff in the air passageway.
XX. The method of paragraph WW, further comprising clamping the tubing at a point beyond a bronchoscope tip after the bronchoscope is removed from the air passageway to ensure the inflatable cuff remains inflated after the bronchoscope is removed from the air passageway.

Although the present disclosure has been shown and described with reference to the foregoing operational principles and embodiments, it will be apparent to those skilled in the art that various changes in form and detail may be made without departing from the spirit and scope of the invention. The present disclosure is intended to embrace all such alternatives, modifications and variances that fall within the scope of the appended claims.

It is believed that the disclosure set forth above encompasses multiple distinct embodiments with independent utility. While each of these embodiments has been disclosed in its form, the specific embodiments thereof as disclosed and illustrated herein are not to be considered in a limiting sense as numerous variations are possible. The subject matter of the present disclosure includes all novel and non-obvious combinations and sub-combinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

The present disclosure embodied in various combinations and sub-combinations of features, functions, elements, and/or properties may be claimed through presentation of new claims in a related application. Such new claims, whether they are directed to a different invention or directed to the same invention, whether different, broader, narrower or equal in scope to the original claims, are also regarded as included within the subject matter of the present disclosure.

1. A medical device for performing a bronchoalveolar lavage (BAL), comprising:
a tubing having
a first lumen configured for communication with a first reservoir, the first lumen having an open distal end configured for communication with the first reservoir,
a second lumen that is separate from the first lumen and configured for communication with a second reservoir, and
an inflatable cuff that is disposed proximate the open distal end and configured for communication with the second reservoir via the second lumen, wherein the open distal end and the inflatable cuff in a deflated position are both configured for receipt within a working channel of a bronchoscope.

2. The medical device of claim 1, wherein the first reservoir is a fluid source housing fluid and the second reservoir is an air source housing air.

3. The medical device of claim 1, wherein a portion of the tubing including the open distal end and the inflatable cuff is sized to be housed within the working channel of the bronchoscope while the cuff is in the deflated position.

4. The medical device of claim 1, further comprising one or more distance markers disposed on the tubing, wherein the markers are disposed on the tubing on a proximal side of the inflatable cuff in a direction toward a proximal end of the tubing.

5-6. (canceled)

7. A method for performing a bronchoalveolar lavage (BAL), comprising:
inserting a movable tubing with an inflatable cuff in a deflated position into an air passageway of a subject;
positioning the movable tubing in the air passageway by visually monitoring an image of a distal portion of the tubing inserted into the air passageway;
insufflating the inflatable cuff to an inflated position to create a wedge to seal off an area of the air passageway; and
aspirating a lavage fluid specimen from the sealed-off area through the tubing.

8. The method of claim 7, further comprising instilling fluid into the sealed-off area through the tubing before aspirating the lavage fluid specimen.

9. The method of claim 8, further comprising instilling approximately 20 milliliters (ml) to approximately 40 ml of fluid and repeating the instilling and aspirating steps at least 4 times.

10. The method of claim 8, further comprising inserting the movable tubing into the air passageway of the subject within a working channel of a bronchoscope and positioning the bronchoscope in the air passageway.

11. The method of claim 10, further comprising extending the inflatable cuff in the deflated position to a desired position beyond a positioned distal tip of the bronchoscope and insufflating the inflatable cuff, which is disposed proximate a distal end of the tubing, to the inflated position to create the wedge.

12. The method of claim 11, further comprising using a camera associated with the bronchoscope to confirm proper positioning of the cuff in the air passageway, the camera transmitting a still image or video image for visual monitoring.

13. The method of claim 10, further comprising deflating the cuff and completely retracting the distal end of the tubing and the cuff into the working channel from the air passageway before removing the bronchoscope after aspiration.

14. The method of claim 10, further comprising extending a first depth marker of a plurality of depth markers on the tubing from inside the working channel to a distal position outside the working channel after the cuff is extended outside the working channel.

15. The method of claim 14, further comprising using a camera to observe the first depth maker to confirm proper positioning of the cuff in the air passageway.

16. A method for performing an endobronchial balloon tamponade of pulmonary hemorrhage, comprising:
inserting a bronchoscope with a working channel housing a movable tubing into an air passageway of a subject;
positioning the bronchoscope in the air passageway;
extending the tubing beyond a positioned distal tip of the bronchoscope to a desired position in the air passageway; and
insufflating in the air passageway an inflatable cuff positioned proximate a distal end of the tubing.

17. The method of claim 16, further comprising positioning the bronchoscope in a bleeding segment or subsegment of the air passageway and extending the tubing to the desired position in the bleeding segment or subsegment.

18. The method of claim 16, further comprising deflating the inflatable cuff to check for hemostasis using a camera of the bronchoscope.

19. The method of claim 16, further comprising removing the bronchoscope from the air passageway while leaving the tubing and the insufflated cuff in the air passageway.

20. The method of claim 19, further comprising clamping the tubing at a point beyond a bronchoscope tip after the bronchoscope is removed from the air passageway to ensure the inflatable cuff remains inflated after the bronchoscope is removed from the air passageway.

21. The method of claim 7, further comprising: providing the movable tubing having a first lumen configured for communication with a first reservoir, the first lumen having an open distal end configured for communication with the first reservoir, a second lumen that is separate from the first lumen and configured for communication with a second reservoir, and the inflatable cuff that is disposed proximate the open distal end and configured for communication with the second reservoir via the second lumen, wherein the open distal end and the inflatable cuff in the deflated position are both configured for receipt within a working channel of the bronchoscope.

22. The method of claim 21, wherein:
insufflating the inflatable cuff to the inflated position is by insufflating air or fluid from the second reservoir through the second lumen, and
aspirating the lavage fluid specimen from the sealed-off area occurs through the first lumen.
